(19) United States
(12) Patent Application Publication
(10) Pub. No.: US 2016/0243146 A1
(43) Pub. Date: Aug. 25, 2016

(54) LEVIA : A New Injectable Parasiticidal Veterinary Formulation of Levamisole HCl and Ivermectin Solution.

(71) Applicant: OTHMAN ABDULRAHIM RADI ALHANBALI, Sydney (AU)

(72) Inventor: OTHMAN ABDULRAHIM RADI ALHANBALI, Sydney (AU)

(21) Appl. No.: 15/010,016

(22) Filed: Jan. 29, 2016

(30) Foreign Application Priority Data
Feb. 21, 2015 (AU) 2015900614

(51) Int. Cl. A61K 31/7048; A61K 9/00; A61K 47/10; A61K 31/429

(52) U.S. Cl. CPC A61K 31/7048; A61K 31/429; A61K 9/0019; A61K 47/10

(57) ABSTRACT

This invention (LEVIA) is a new preservative free veterinary formulation, composed of 7.5% Levamisole HCl and 1% Ivermectin. This unique combination provides a very effective anthelmintic effect against, animal pests and endoparasites. In addition, this formula may be used for combating, killing and/or controlling:
1—Adult and immature gastro intestinal round worms;
2—Lung worms, eye worms and warble fly larvae;
3—Helps in treatment of Sarcoptes and Psoroptes Scabies (mange mites) and control of Sucking Lice.

This invention provides an improved method for the treatment of helminthiasis in mammals, particularly in: bovines, caprines, equines, ovines, canines and felines.

### FIELD OF THE INVENTION

The present invention concerns a veterinary, preservative free injectable pharmaceutical combination of 7.5% Levamisole HCl and 1% Ivermectin.

This anthelmintic formulation has been outstanding for several months now for veterinary treatment, in particular for cattle, sheep and horses, in view of its good. performance and therapeutic harmlessness in eliminating and/or regulating both internal and/or external parasites.

The chance for this new anthelmintic drug to make it to market is very likely.

### BACKGROUND OF THE INVENTION

Resistance of gastro-intestinal nematodes to anthelmintic agents constitutes a major threat to the worldwide small ruminant industry.

Reports demonstrate the occurrence of anthelmintic resistance in animal pests and endoparasites, and present evidence of lack of efficacy has been reported for various types of worms preventatives in the USA, UK and EU.

The most salient feature of anthelemantic resistant is that it is inherited. It is populations of worms—and not the individuals—that become resistant.

Despite the palpable need for new classes of antihelmintics to counter the mounting threat of resistance, the stream of new drugs delivered in the livestock endoparasiticide market has been insufficient.

Unfortunately, various combinations anthelmintics of other families have proven. difficult to formulate at high concentration and with an acceptable viscosity for injection.

To overcome the problem of resistance, various combinations of anthelmintics have been extensively researched and this innovation may launch a new anthelmintic combination class for use in animals.

This innovation, with a single and/or multiple administration of 7.5% Levamisole Hydrochloride+1% Ivermectin, will kill or control the parasites present in the host at the time of treatment, and this formulation will provide treatment for parasitic infections and act as immunomodulatory agent on the immune system.

This invention regulates the development of a resistant parasite inhibatant by lowering the number of resistant genotypes, which survive treatment, since manifold alleles conferring resistance to all the component anthelmintic classes must be present in the same parasite for survival.

Animals carrying multiple resistance alleles are rarer than those carrying single resistance alleles.

This enhanced efficacy leads to greater dilution of resistant genotypes by the unselected parasites in refugia, thus reducing the proportion of resistant parasites available to reproduce with other resistant adults that have survived treatment.

Ivermectin has a very poor solubility in water, at a level of about 0.005 mg per ml at room temperature, and is unstable in water. On the other hand, it is known that Ivermectin dissolved in propylene glycol solvent releases into the blood during a period of up to 14 days. Reference may be made to European Patent (EP) application 0045 655 (filed on 10 Feb. 1982) which discloses a preparation of Ivermectin in glicerol-formal and propylene glycol to increase the duration of the pesticide activity.

Ivermectin is very soluble in many organic solvents, in this invention Propylene glycol, offers a useful liquid medium for injection compare to the market available preserved anthelmantic analogues were micelles are used.

The use of this invention would be better for anthelmintic management in animals, as no preservative or any other excipients are used, this makes this formulation:
1—Long acting anthelmintic activity (at least 14 days)
2—Safer than other anthelmintic drugs in the market
3—More stable formulation
4—Economically feasible

### SUMMARY OF THE INVENTION

This invention relates to a new injectable pharmaceutical anthelmintic formulation, composed of combinations of 7.5% Levamisole HCl and 1% Ivermectin for veterinary treatment.

This new formula designed with a related anthelmintic spectrum of activity and different mechanisms of action on anthelmintic resistance, this invention combines anthelmintics from different classes reduces the potential of parasites to survive the treatment.

In view of its good performance and therapeutic harmlessness in eliminating both internal and external parasites, this invention is suitable to be released to market.

The main reason for using such combinations:

To enable more effective and control of adult and immature gastro intestinal round worms in the presence of single or multiple drug resistance;

To provide efficient treatment for parasitic infections and act as immunomodulatory agent on the immune system for various animals;

To slow the development of resistance to the component anthelmintic classes by using this new preservative free formulation.

### BRIEF DESCRIPTION OF THE INVENTION

The instant invention concerns the preparation of an 7.5% Levamisole Hydrochloride+Ivermectin parenteral formulation, in particular, the aim of using combinations of anthelmintics from different classes to reduce the potential of parasites to survive the treatment and to minimize the ability of animal pests and/or other endoparasites to resist the effects anthelmintic agent.

In this new anthelmintic agent, using propylene glycol formal and water for injection, is ideal for chemical, physical, economical and environmentally friendly formulation.

This invented formula is designed, to avoids many problems associated with preservative and/or excipients—active material incompatibility. In additions, this formulations to benefit from its ultra-high purity, stability and avoidance of preservatives, or other additives, thus assuring excellent formulation compatibility and maximum stability.

This new formulation with water at a lower concentration to achieve the maximum protection profile of activity for the Levamisole HCl and Ivermectin by providing minimal mcrobial growth media. Thus, it is an object of this invention to describe such a formulation.

A further object is to describe the solvents used Propylene glycol. Where Propylene glycol is a form of mineral oil, an alcohol produced by fermentation of yeast and carbohydrates. This gives it the designation of carbohydrate when used in foods and pharmaceutical.

The FDA includes Pharmaceutical grade Propylene glycol on its Generally Recognized As Safe (GRAS) list. The World Health Organization also considers it as safe for use.

### DETAILED INFORMATION FOR THIS NEW PROPOSED PRODUCT

Composition: Per 100 ml. solution: 7.5% Levamisole HCl and 1% Ivermectin injection.

Appearance: Colorless clear sterile solution, slightly viscous.

Description: Levamisole HCl and Ivermectin, are parasiticide for the treatment and control of internal and external parasites , such as gastro-intestinal roundworms, lungworms, grubs, screwworms, fly larvae, lice, ticks and mites in cattle, sheep, goats pigs and camels camels.

Specification: Each 100 mL contains 7.5 g Levamisole HCl and 1 g Ivermectin, preservative free solution.

Instructions:
a—For veterinary use only
b—Do not smoke or eat while handling the product
c—Wash hands before and after use
d—Keep out the reach of children
e—The container and residue of this drug must be disposed safely and particularly the drug is extremely toxic to fish and other species of wild aquatic

Route of administration: Exclusively by Subcutaneous

Contra-indications: This production must not be used intramuscularly or intravenously

Side-effects: Soft tissues swelling at the injection site has been observed.

Withdrawal period:
a—Animals must NOT be slaughtered for human consumption during treatment.
b—Animals may be slaughtered for human consumption only after 35 days from the last treatment.
c—Milk for human consumption must NOT be taken during treatment. Milk for human consumption may only be taken from treated Animals after 30 days from the last administration.

Package: 100 mL/Amber vial; 500 mL/Amber vial

Storage: Store sealed in cool, dry, shady place

Period of validity: 3 years

Specifications:
a—7.5% Levamisole HCl and 1% Ivermectin injection
b—Antiparasitic drugs
c—Colorless liquid
d—Preservative free injectable
e—Liquids used: Propylene glycol up to 93% and water for injection is 7%
f—Pyrogen free injection
g—Stable characters
h—Highly effective

### Tests

The effectiveness of this new invention was invivo tested on various types of animals (sheeps, cows, goats and horses) and the evaluation was focused on killing and /or expulsion or reducing number of certain types of worms and Sucking Lice from their hosts.

The reduction in the number of eggs per gram of feces passed by the infected hosts and then compared with commercial anthelmintic treated animals .

Over 100 sheep and 30 cows calves populations tested over a period of UP TO 6 MONTH at the recommended dose rate mentioned in claims 2 and 3; this invention which is comprises from the combinations of 7.5% Levamisole Hydrochloride+1% Ivermectin; gives appropriate therapy way for adult and immature gastro intestinal round worms and Sucking Lice and managed resistance.

The results showed a significant effect on treated animals with efficacy of 97.75% in eliminating both adult and/or immature gastro intestinal round worms, lung worms, eye warms and warble larvae.

All treated animals were monitored for 6 months after complete recovery to observe if any were re-infected, or the animals were developing resistance against this drug.

The results were 98.47% free from any parasite 6 months post treatment with this invention.

### Stability Test

Stability studies were carried out to ensure the maintenance of final product quality, safety and efficacy throughout the shelf life of 3 years.

What is claimed is:

1. LEVIA is a new colorless, preservative free sterile veterinarian solution, adapted to be subcutaneously injected into animals, this formulation comprises of:
a. an effective amount of 7.5% Levamisole HCl;
b. an effective amount of at least 1% Ivermectin;
c. a pharmaceutically acceptable system of Propylene glycol C3H802 (mwt 76.1) as a solvent wherein the system provides for acceptable storage stability for both the Levamisole HCl and the Ivermectin and wherein the system provides for a viscosity which is suitable for injection; and wherein the system contains 7% water for injection.

2. The formulation of claim 1, wherein 7.5% Levamisole HCl salt is present in an amount sufficient to deliver a maximum dose of 2 mL where each one mL contains 75 mg/mL and the recommended dose between 1 to 2 mL/50 kg chattels bodyweight. A second and final dose could be needed after 21 days from the first dose.

3. The formulation of claim 1, wherein the 1% Ivermectin is present in an amount sufficient to deliver a dose of at least 10 mg/ml. Each one mL contains 10 mg/mL and the recommended dose is between 1 to 2 mL/50 kg animal bodyweight. A second and final dose could be needed 21 days from the first dose

4. The formulation of claim 1, wherein the solvents used are propylene glycol and water for injection, where 7% of the liquid used in this invention is water for injection and the remaining to make up the total volume is propylene glycol.

5. The formulation of claim 1 contains no preservative, no additives or any other excipients.

6. This subcutaneous solution is pyrogen free and sterile.

7. The formulation of claim 1 wherein the Levamisole HCl be an effective amount of at least 7.5% and Ivermectin its concentration is about 1% (w/v); the solvent is Propylen glycol and its concentration is between 90-93% (v/v), and this percentage acts as a preservative for this formulation, and water for injection is used as remaining quantity to make up the total volume, which is 7% (v/v).

8. This new formulation of any one of claims 1-5 wherein the 7.5% Levamisole HCl (w/v) and wherein the 1% Ivermectin (w/v) concentration has a unique physical specifications, such as; The density of the final formulation is 1.05±0.004 at 28° C., pH is around 3.50±0.5, kinetic viscosity 23.90±1.08 milli pascal-second (mPa·s) and 25.07±1.17 (mPa·s).

9. Levia is a method for preventing or treating internal parasite infestations in animals, which comprises of parentral administration to an animal, with an effective amount of the formulation of claim 1.

10. The method of claim 8 wherein the formulation is injected subcutaneously.

11. The method of claim 9 wherein the parasites are Adult and Immature gastro intestinal round worms, lung worms, eye worms and warble larvae.

12. The method of claim 10, the Sucking Lice and mange mites (Sarcoptes and Psoroptes Scabies).

13. The method for producing the formulation of claim 1, comprising the steps of:
a. Dissolving the 1% Ivermectin with a suitable solvent, which is propylene glycol. This solvent represents around 92% from the whole formula.
b. In a separate vessel, adding and dissolving 7.5% Levamisole HCl in known amount of water for injection, about (7.0%).
c. Adding Levamisole HCl solution to Ivermectin bulk solution with mixing speed of (150 rpm at 30° C. in this step only).
d. Optionally, bringing the batch to Total volume with added propylene glycol and mixing; thereby producing the formulation.
e. Temperature must be maintained at 25° C. in all steps except step 13d.
f. The final formulation filled under aseptic conditions and nitrogen gas and then packaged into an air-tight container suitable for injection. This should stay suitably preserved through the shelf life of the product, which is around three years.
